(19) United States
(12) Patent Application Publication
(10) Pub. No.: US 2006/0067995 A1
(43) Pub. Date: Mar. 30, 2006

(54) Topical and/or transdermal bioactive compound delivery system

(76) Inventors: Wan Sia Paul Heng, Singapore (SG); Jinsong Hao, Singapore (SG); Lai Wah Chan, Singapore (SG); Ma Shwe Zin Nyunt, Singapore (SG); Gil Yosipovitch, Winston Salem, NC (US); Yolande Anthony, Singapore (SG)

Correspondence Address: OSTROLENK FABER GERB & SOFFEN, 1180 AVENUE OF THE AMERICAS, NEW YORK, NY, 100368403, US

(21) Appl. No.: 11/173,536

(22) Filed: Jul. 1, 2005

Related U.S. Application Data
(60) Provisional application No. 60/584,666, filed on Jul. 2, 2004.

(51) Int. Cl. A61K 9/70

(52) U.S. Cl. 424/449

(57) ABSTRACT

Topical and/or transdermal bioactive compound delivery system The present invention provides a topical and/or transdermal bioactive compound delivery system comprising a non-aqueous matrix, the matrix comprising a water-sensitive bioactive compound. In particular, the matrix comprises a three-dimensional polymeric network comprising at least a co-polymer, at least a cross-linking agent, and at least a non-aqueous and/or non-volatile solvent and wherein the water-sensitive bioactive compound is housed by the three-dimensional network. Even more in particular, the matrix comprises a co-polymer of N-vinylacetamide and sodium acrylate aluminium chloride; N-methyl-2-pyrrolidone; propylene glycol; glycerine; and at least a water-sensitive bioactive compound.

### CROSS-REFERENCE TO RELATED APPLICATION

This application claims the benefit of U.S. Provisional Application No. 60/584,666, filed on Jul. 2, 2004, the entirety of the contents of which are hereby incorporated by reference herein.

### FIELD OF THE INVENTION

The present invention relates to a topical and/or transdermal bioactive compound delivery system. In particular, to a bioadhesive delivery system. More in particular, to non-aqueous compositions for topical and/or transdermal delivery of water-sensitive drugs.

### BACKGROUND OF THE INVENTION

Topical and/or transdermal drug delivery systems have been widely recognized in achieving dermatological drug therapy and systemic pharmacological actions. The conventional topical and/or transdermal drug delivery systems are formulated in the form of patches/plasters, creams, ointments, gels and solutions. Creams, ointments, gels and solutions are mainly used in topical application. However, it is relatively difficult for the patient to accurately control the drug amount administered to the site of application. Moreover, the compliance is usually poor because of the sticky feeling and soiling of clothes due to these dosage forms. The incorporation of volatile solvents such as ethanol and water in these formulations usually leads to the change of the composition or growth of microbial after long term storage. The presence of water in the formulations also influences the stability of water sensitive drugs.

Patches and/or plasters are more compliant and acceptable compared to the semi-solid and liquid preparations. They are useful not only for topical drug application but also for systemic drug administration. However, the existing technologies exhibit some disadvantages associated with the use of pressure sensitive adhesives (PSAs) in the fabrication of these dosage forms to allow the dosage forms to adhere on skin for a long period of time. Patches and/or plasters are basically categorized into two types i.e. matrix type device and reservoir type device. Irrespective of the type, PSAs are important ingredients of patch and/or plaster formulations. Reservoir type devices typically consist of a reservoir filled with a liquid preparation of the active ingredient and a controlled release membrane, exhibiting suitable adhesive properties as well. This type of system can load a relatively large amount of drug but dose dumping due to leakage from the reservoir is another disadvantage and the use of controlled release membrane also complicates the fabrication process. Matrix type devices are new development in transdermal drug systems, which consist, in the simplest form, of three layers i.e. a backing layer, a drug loaded adhesive layer and a protection layer. The drugs and additives are dissolved or dispersed in the adhesive matrix, depending on the amount added for the appropriate delivery profile. Low drug capacity is a limitation besides the problems due to PSAs.

The acrylic class of adhesives dominates the medical PSAs market because they possess good adhesive properties on the skin and are available in a wide variety of compounds with different properties with respect to their physical and chemical properties. However, it is well known that these PSAs are essentially hydrophobic and are not suitable, in some cases, to be loaded with a hydrophilic polar drug, for long-term wear and wound care. They usually lose their adhesive properties in the presence of moisture and thus, excessive perspiration of the skin is a detrimental factor to adhesive property of PSAs.

Moreover, active ingredients and other additives incorporated into the adhesive in the matrix type systems can greatly influence the cohesive and adhesive properties. Dissolved additives tend to decrease adhesion and render the adhesive more susceptible to creep/cohesive failure. The cohesive failure of the adhesive matrix during storage then leads to occurrence of cold flow, in which the adhesive matrix begins to flow beyond the backing layer and eventually sticks to the primary packaging container and thus, the plasters become unusable. The cohesive failure also causes adhesive residues to remain at the application site when the plaster is removed from the skin. These problems become particularly evident when the concentration of additives dissolved in the adhesives increases. On the other hand, dispersed additives tend to reinforce the adhesive, especially if the additive is a solid. In the matrix type system, drugs are usually dispersed in the adhesive matrix due to the limited capacity of the adhesive matrix to accommodate drugs, in which thermodynamic activity of drugs is not maximized and thus the permeation of the drug through the skin is compromised. The conventional systems have been extensively used for delivery of potent drugs with low daily dose but the delivery of high dose of drugs is greatly limited.

In order to overcome cold flow problems associated with PSAs, U.S. Pat. No. 6,436,433 discloses a transdermal or topical plaster system with a polyacrylate matrix with improved physical properties. The system consists of at least a two-layer matrix, with each layer having the same polymer composition and dissolved ingredients but different cross-linking degrees, where the layer facing away from the skin has a degree of cross-linking which is sufficient to prevent cold flow, and the layer facing the skin has a degree of cross-linking which ensures adequate adhesion to the skin. The two adhesive layers are prepared separately and laminated after removal of the organic solvents.

Transdermal therapeutic system for administering steroidal hormones produced by a solvent-free melt extrusion method is disclosed in U.S. Pat. No. 6,562,367. The active ingredients are incorporated continuously into the hot polymer melt heated up to 200° C., the hot drug-containing polymer melt is then coated directly onto a separable protective layer. Unfortunately, the drug is subject to a high temperature, which is a great challenge to drug stability. U.S. Pat. No. 6,268,355 discloses stable aspirin containing preparations for external use, in which at least one substance selected from an ester of an organic acid ester having 2 to 20 carbon atoms, a glycerol fatty acid ester, silicon oil, hydrocarbon oil and crotamiton are used to dissolve and stabilize aspirin. Obviously, other excipients have to be used for formulating the invention in different types of external preparations, such as cataplasms, plasters, ointments, creams and external powders, etc.

There is therefore a need for improved and/or alternative drug delivery systems which allow good adhesion to the biological tissues and delivery of high dose of drug(s).

### SUMMARY OF THE INVENTION

The present invention addresses the problems above and provides a topical and/or transdermal bioactive compound delivery system, as well as a method for delivery of bioactive compound(s). In particular, the invention provides a bioadhesive topical and/or transdermal system for delivery of bioactive compound(s). More in particular, a bioadhesive topical and/or transdermal drug-delivery system.

According to one aspect, the present invention provides a topical and/or transdermal bioactive compound delivery system comprising a non-aqueous matrix, the matrix comprising a water-sensitive bioactive compound.

In particular, the matrix is prepared by mixing: at least one polymer, at least one cross-linking agent, and at least one non-aqueous and/or non-volatile solvent, and wherein the non-aqueous and/or non-volatile solvent comprises the bioactive compound.

The matrix may comprise a three-dimensional polymeric network comprising at least a co-polymer, at least a cross-linking agent, and a non-aqueous and/or non-volatile solvent, and wherein the water-sensitive bioactive compound is housed by the three-dimensional network. The three-dimensional network may be a hydrogel.

The polymer may be a polymer or co-polymer selected from, but limited to, a polymer of N-vinylacetamide, a polymer of acrylic acid, a polymer of acrylate, a co-polymer of acrylic acid and sodium acrylate. The cross-linking agent may be an aluminium salt or a trivalent salt. In particular, the cross-linking agent is aluminium chloride. The non-aqueous and/or non-volatile solvent may be N-methyl-2-pyrrolidone. The bioactive compound may be a drug. In particular, the drug is aspirin.

According to a particular, aspect, the delivery system is prepared by mixing: (a) a co-polymer or N-vinylacetamide and sodium acrylate; (b) aluminium chloride; (c) N-methyl-2-pyrrolidone; (d) propylene glycol; (e) glycerine; and (f) at least a water-sensitive bioactive compound.

According to a particular aspect, the present invention provides a bioadhesive topical and/or transdermal bioactive compound delivery system comprising a non-aqueous matrix, wherein the matrix is prepared by mixing (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

According to another aspect, the invention provides a bioactive compound delivery composition which is made of a water-insoluble polymeric networks capable of imbibing a large amount of solvents to accommodate a high dose of bioactive compound.

According to another aspect, the present invention provides a non-aqueous bioactive compound delivery composition which is made up of non-volatile solvents such that it is suitable for delivery of water-sensitive drugs without change of composition over time.

According to another aspect, the present invention provides a bioactive compound delivery composition which exhibits bioadhesive properties. It can stick to skin for a longer period of time but upon removal, no residues are left on the skin.

According to another aspect, the present invention provides plasters which show little or no irritation after long-term application.

In particular, the bioactive compound may be at least one drug, and the delivery system is a drug delivery system.

In particular, the delivery system according to the invention is a bioadhesive topical and/or transdermal delivery system.

The delivery system according to any embodiment of the invention may be in the form of a plaster or patch. The plaster may comprise a backing layer, wherein the backing layer comprises at least one material selected from polyester, polypropylene and polyethylene.

According to another aspect of the present invention, it is provided a method of preparing a topical and/or transdermal bioactive compound delivery system comprising preparing a polymeric non-aqueous matrix, the matrix comprising a water-sensitive bioactive compound.

The matrix may be prepared by mixing: at least one polymer, at least one cross-linking agent, and at least one non-aqueous and/or non-volatile solvent, and wherein the non-aqueous and/or non-volatile solvent comprises the bioactive compound.

According to a particular aspect, the matrix is prepared by mixing (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

According to another aspect, the present invention provides a method of topical and/or transdermal administration of a water-sensitive bioactive material, comprising preparing a polymeric non-aqueous matrix, the matrix comprising a water-sensitive bioactive compound, and applying the matrix on the skin of a mammal in need of treatment.

The matrix may be prepared by mixing: at least one polymer, at least one cross-linking agent, and at least one non-aqueous and/or non-volatile solvent, and wherein the non-aqueous and/or non-volatile solvent comprises the bioactive compound.

According to a particular aspect, the matrix is prepared by mixing (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

The bioactive compound described according to any aspect of the invention may be a drug.

According to a particular aspect, the topical and/or transdermal administration is in the form of a plaster or patch.

Accordingly, bioadhesive, non-aqueous compositions for topical or transdermal delivery of bioactive compounds with characteristic of water-sensitivity and high dose are described which comprise (a) co-polymer of N-vinylacetamide and sodium acrylate, or its equivalents, (b) aluminium chloride, or its equivalents, (c) N-methyl-2-pyrrolidone, or its equivalents, (d) propylene glycol, or its equivalents, (e) glycerine, and its equivalents, and (f) a drug.

Particularly, these compositions fabricated in the form of plasters are prepared by cross-linking copolymer of N-vinylacetamide and sodium acrylate with aluminium chloride in the presence of non-aqueous solvents such as N-methyl-2-pyrrolidone, propylene glycol and glycerine. Active ingredients are dissolved in the solvents which are entrapped in the microcavities of three-dimensional networks formed. These plasters exhibit bioadhesive properties and have a great capacity to accommodate a high dose of drugs. The non-aqueous nature of the compositions is suitable for delivery of water-sensitive drugs. A mode water-sensitive drug used is aspirin.

### BRIEF DESCRIPTION OF THE FIGURES

FIG. 1 is a graphical representation of percentage of drug released vs. time in pH 7.4 phosphate buffer at 32° C. from Examples 1-13 through filtration membrane.

FIG. 2 is a graphical representation of cumulative amount of drug permeated through porcine skin epidermis vs. time in pH 7.4 phosphate buffer solution at 32° C. from Examples 1- 13.

FIG. 3 is a graphical representation showing the irritation results obtained from LDPI after application of Examples 1-16 on porcine skins for 14 days. LDPI values after application of placebo plasters (Example IC to Example 13C) on porcine skins for 14 days are also illustrated in the graphs for comparison.

FIG. 4 is a graphical representation showing detachment forces of Examples 1-13 to a model skin-mimic substrate, silicone elastomer.

FIG. 5 is a graphical representation of loss tangent values versus oscillatory frequency in Examples 1-13, indicating the compositions are cross-linked networks in nature.

### DETAILED DESCRIPTION OF THE INVENTION

The whole content of any bibliographic references mentioned in the present specification is herein incorporated by reference.

Bioadhesion may be defined as the ability of polymers to adhere to biological tissues in the presence of a solvent.

Hydrogels are three-dimensional, hydrophilic, polymeric networks capable of imbibing a large amount of water and water miscible solvents. The high solvent content of hydrogels can provide a great capacity to accommodate a high dose of drugs and good compliance compared to conventional dosage forms such as ointments, creams, gels and patches. Therefore, bioadhesive hydrogels formulated in proper dosage forms, e.g. plasters, will overcome the above-mentioned problems associated with the conventional systems. The bioadhesive hydrogels are promising in the development of drug delivery systems for percutaneous or mucosal application. Since the bioadhesive polymer molecules are cross-linked with an appropriate cross-linking agent in the matrix, they are not sticky but still maintain their bioadhesive properties. Further, hydrogels have a good compatibility with the skin and mucosal membranes.

Accordingly, the present invention provides a topical and/or transdermal bioactive compound delivery system comprising a non-aqueous matrix, the matrix comprising a water-sensitive bioactive compound. In particular, the invention provides a bioadhesive topical and/or transdermal bioactive compound delivery system comprising a non-aqueous matrix, the matrix comprising a water-sensitive bioactive compound.

The matrix may be prepared by mixing: at least one polymer, at least one cross-linking agent, and at least one non-aqueous and/or non-volatile solvent, and wherein the non-aqueous and/or non-volatile solvent comprises the bioactive compound.

Further, the matrix may comprise a three-dimensional polymeric network comprising at least a co-polymer, at least a cross-linking agent, and at least a non-aqueous and/or non-volatile solvent, and wherein the water-sensitive bioactive compound is housed by the three-dimensional network. The three-dimensional network may be a hydrogel.

The polymer or co-polymer according to any aspect of the present invention includes, but is not limited to, polymer of N-vinylacetamide, polymer of acrylic acid, polymer of acrylate, co-polymer of acrylic acid and sodium acrylate.

The cross-linking agent may be an aluminium salt or a trivalent salt. In particular, the cross-linking agent is aluminium chloride.

The non-aqueous and/or non-volatile solvent may be N-methyl-2-pyrrolidone.

Accordingly, the matrix of the present invention is prepared by mixing: (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound. The bioactive compound may be a drug. In particular, the drug is aspirin.

According to a particular aspect, the delivery system is a bioadhesive topical and/or transdermal delivery system. In particular, the delivery system is in the form of a plaster. The plaster may comprise a backing material, wherein the backing material comprises at least one of the following materials: polyester, polypropylene and polyethylene. The backing material may be a non-woven cloth or a release liner.

The advantage of a topical delivery system is that high local tissue levels are achievable, higher than that by systemic route. It is also a less toxic delivery system. Examples of topical delivery systems include ointments, pastes, gels, medicated powders, creams, lotions, aerosols, sprays, foams and medicated adhesives. Transdermal delivery systems allow sustained delivery of a drug over days and often at a constant drug-release rate, with the delivery rate typically being zero-order. Further, the composition is usually invariant in use. The system size is reasonable and it provides a defined site for application. The application technique may be highly reproducible.

The present invention also provides a bioactive compound delivery composition which is made of water-insoluble polymeric networks capable of imbibing a large amount of solvents to accommodate a high dose of bioactive compound.

According to another aspect, the present invention provides a non-aqueous bioactive compound delivery composition which is made up of non-volatile solvents such that it is suitable for delivery of water-sensitive drugs without a change of composition over time.

According to another aspect, the present invention also provides a bioactive compound delivery composition which exhibits bioadhesive properties. It may be able to stick to the skin for a longer period of time but upon removal, no residues are left on the skin.

Accordingly, the present invention provides plasters which show little or no irritation after long-term application.

In accordance with this invention, bioadhesive, non-aqueous compositions for topical or transdermal drug delivery are provided which mainly consist of (a) co-polymer of N-vinylacetamide and sodium acrylate, or its equivalents, (b) aluminium chloride, or its equivalents, (c) N-methyl-2-pyrrolidone, or its equivalents, (d) propylene glycol, or its equivalents, (e) glycerine, or its equivalents, and (f) a drug, drugs or therapeutic agents.

The co-polymer of N-vinylacetamide and sodium acrylate (PNVA GE-167, Showa Denko K.K, Japan) exhibits non-ionic properties of its monomer of N-vinylacetamide, as well as weak anionic characteristic of its monomer of sodium acrylate. This special structure renders the polymer some typical properties. For example, the polymer is readily soluble in water, glycerine and various organic solvents, especially ethanol aqueous solutions. In the presence of a trivalent ion, the polymer is cross-linked and forms an elastic hydrogel. The hydrogel formed is capable of holding a large amount of solvents within the microcavity of the three-dimension network structure of the hydrogel, which is a favourable feature for loading a high dose of drugs. The polymer molecules in the network structure maintain their bioadhesive properties but are not sticky due to the nature of the cross-linked structure. These properties make it possible to formulate a bioadhesive non-aqueous delivery system of a water-sensitive drug at a high dose.

Accordingly, another aspect of the present invention provides a bioadhesive topical and/or transdermal bioactive compound delivery system comprising a non-aqueous matrix, wherein the matrix is prepared by mixing (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound. In particular, the delivery system is in the form of a plaster or patch. The plaster may comprise a backing layer, wherein the backing layer comprises at least one material selected from polyester, polypropylene and polyethylene.

In particular, the bioactive compound may be at least one drug, and the delivery system is a drug delivery system. According to a particular aspect, the present invention provides a method of preparing a topical and/or transdermal bioactive compound delivery system comprising preparing a polymeric non-aqueous matrix, the matrix comprising a water-sensitive bioactive compound.

According to another aspect, the present invention provides a method for preparing a topical and/or transdermal bioactive compound delivery system comprising a water-sensitive bioactive compound.

The matrix may be prepared by mixing: at least one polymer, at least one cross-linking agent, and at least one non-aqueous and/or non-volatile solvent, and wherein the non-aqueous and/or non-volatile solvent comprises the bioactive compound.

In particular, the matrix is prepared by mixing (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound. The bioactive compound may be a drug. In particular, the drug is aspirin.

Accordingly, co-polymer of N-vinylacetamide and sodium acrylate and aluminium chloride are cross-linked in the presence of N-methyl-2-pyrrolidone, propylene glycol and glycerine at an appropriate amount and weight ratio. The compositions are formulated in suitable dosage forms, such as plasters, by a casting evaporation technique. Some dispersing agents, for example alcohol, facilitate the preparation process, which are removed from the matrix by drying the compositions in a desiccator. The plasters may include a backing layer such as a non-woven cloth and a release liner, which may be made of any suitable material, such as polyester, polypropylene, polyethylene or other films which may function as a backing.

The polymer is dispersed in N-methyl-2-pyrrolidone containing a suitable amount of drug. Propylene glycol is then added to get a uniform drug-polymer solution. The obtained solution is then diluted with an appropriate amount of ethanol and cast in a container. The casting solution in the container is allowed to level off before the aluminium chloride-ethanol solution is added to cross-link the polymer molecules. A certain amount of glycerine is added onto the layer of aluminium chloride-ethanol solution, which retards the evaporation of ethanol and ensures the diffusion of aluminium chloride and complete cross-linking with the polymer molecules. Upon completion of the cross-linking process, the matrix in the container is dried in a desiccator. The dried plaster facing upwards is laminated with a non-woven backing and then covered. The plaster facing downwards is laminated with a release liner. The release liner is removed upon application of the plaster on the skin. The polymer amount and weight ratio of polymer to cross-link agent are critical factors in determining the physical properties in terms of bioadhesion, flexibility, homogeneity, gel strength, aesthetics and drug release from the compositions. The amount and composition of organic solvents used have predominant effects on permeation and irritation on the porcine skins in addition to the effects on the above-mentioned physical properties.

The delivery system according to any aspect may be clear, non-tacky and homogeneous. The delivery system may be in the form of plaster. A plaster shows greater flexibility and conformability to body contours. Further, the delivery system according to any aspect exhibits desirable bioadhesive properties and may stick to skin for a longer period of time. Further, upon removal, it does not leave residues on the skin on which it was applied.

The matrix comprised in the delivery system is made of relatively non-volatile solvents, thus the composition of the matrix will not change over time. Bioactive compounds, such as drugs, are dissolved in the solvents that are entrapped in the microcavity of the cross-linked networks and are released in appropriate controlled modes from the matrix. The non-aqueous nature of this invention is particularly advantageous for delivering water-sensitive drugs at a high dose. The composition of the matrix of the present invention may also be used for the delivery of suitable biotechnology products such as proteins and/or peptides and even herb extracts that usually have complicated compositions at a high dose.

Equivalents of the polymers mentioned may be used in the preparation of the composition of the matrix. The equivalent of co-polymer of N-vinylacetamide and sodium acrylate include, but is not limited to, polymer of N-vinylacetamide, polymer of acrylic acid, polymer of acrylate, and co-polymer of acrylic acid and sodium acrylate. Alternatives to aluminium chloride include different aluminium salts and trivalent salts. However, the preferred polymer of this invention is a co-polymer of N-vinylacetamide and sodium acrylate (e.g. PNVA GE-167, as above) and the preferred cross-linking agent is anhydrous aluminium chloride. The preferred non-aqueous and/or non-volatile solvents (organic solvents) are N-methyl-2-pyrrolidone, propylene glycol and glycerine.

Accordingly, the matrix according to any aspect of the present invention may be prepared using different grades and amounts of the co-polymer of N-vinylacetamide and sodium acrylate, different weight ratios of the polymer to aluminium chloride and organic solvents. Further, the matrix can be fabricated in a solid or semi-solid form for topical and/or transdermal application with different physical and release properties.

The amount of bioactive compound, such as aspirin, is preferably from 2.5 to 10% of the total weight of the plaster. The concentrations of co-polymer of N-vinylacetamide and sodium acrylate (PNVA GE-167) may vary from 5 to 13% (w/w), preferably 9% (w/w). The weight ratios of co-polymer of N-vinylacetamide and sodium acrylate (PNVA GE-167) to aluminium chloride may be 2.5:1 to 1:1.5, preferably 1.5:1 to 1:1. The weight ratio of propylene glycol and N-methyl-2-pyrrolidone is 4:1 to 1:2, preferably 2:1.

Any suitable drug, which is used in topical and transdermal delivery, may be incorporated into the delivery system as a medicament agent. However, the amount of drug, as well as the polymer grade and ratio in the formulation, can be varied widely, depending on the desired release profile and therapeutic effect of the particular bioactive material or drug. Although the composition of the matrix is primarily designed for percutaneous drug delivery, it may also be used in mucosal delivery of drugs.

According to another aspect, the present invention provides a method of topical and/or transdermal administration of a water-sensitive bioactive material, comprising preparing a polymeric non-aqueous matrix, the matrix comprising a water-sensitive bioactive compound, and applying the matrix on the skin of a mammal in need of treatment.

The matrix is prepared by mixing: at least one polymer, at least one cross-linking agent, and at least one non-aqueous and/or non-volatile solvent, and wherein the non-aqueous and/or non-volatile solvent comprises the bioactive compound.

In particular, the matrix is prepared by mixing (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound. The bioactive compound may be a drug. In particular, the drug is aspirin.

The matrix may comprise a three-dimensional polymeric network. In particular, the bioactive compound may be housed within the three-dimensional polymeric network. The three-dimensional polymeric network may be a hydrogel.

The topical and/or transdermal administration may be in the form of a plaster or patch. The plaster or patch may comprise a backing material, wherein the backing material comprises at least one of the following materials: polyester, polypropylene and polyethylene. The backing material may be a non-woven cloth or a release liner.

While the invention has been described with particular reference to certain embodiments thereof, it will be understood that various modifications can be made to the above mentioned embodiment without departing from the spirit and scope of the present invention. The examples and the particular proportions set forth are intended to be illustrative only.

### EXAMPLES

Preparation of Plaster

Aspirin plasters containing co-polymer of N-vinylacetamide and sodium acrylate (e.g. PNVA GE-167, Showa Denko K.K, Japan) and a cross-linking agent anhydrous aluminium chloride were formulated in the presence of solvents such as N-methyl-2-pyrrolidone under the trade name of Pharmasolve (ISP, USA), propylene glycol and glycerine.

Drug release and permeation through the porcine skins were carried out using Franz diffusion cell system (Microette, Hanson Research, US). The irritation of the plasters on porcine skins was investigated by visual analogue score (VAS) system and Laser Doppler Perfusion Imager (LDPI, Lisca Development AB, Linkoping, Sweden). The physical properties of the compositions such as bioadhesion and rheological properties were also evaluated. The bioadhesive properties of these plasters to a model skin-mimic substrate, silicone elastomer, were measured by using a tensile tester (EZ tester, Shimadzu, Japan) in the tensile mode. The Theological properties were studied using the rheometer (RheoStress® I Haake, Germany).

The plasters with a plastic release liner and non-woven backing layer were prepared by a casting evaporation technique from non-aqueous polymer solutions in the presence of N-methyl-2-pyrrolidone, propylene glycol and glycerine. The amount of aspirin in the present invention was between 2.5 and 10% of the total weight of the plaster. The concentrations of PNVA GE-167 was between 5 and 13% (w/w), preferably 9% (w/w). The weight ratios of PNVA GE-167 to aluminium chloride was between 2.5:1 to 1:1.5. The weight ratio of propylene glycol to N-methyl-2-pyrrolidone was between 4:1 and 1:2. The glycerine of 0.7 g was added into each petri dish with a diameter of 8.5 cm. The amount of aspirin-containing casting solution in each petri dish was between about 3.4 and 4.4 grams and the thickness of dried plasters exclusive of the backing layer and release liner was about 0.5-0.6 mm. The amount of aspirin per square centimeters of plasters ranged from 1.7 to 4.8 mg.

### Example 1

The composition of the matrix consisted of co-polymer of N-vinylacetamide and sodium acrylate (PNVA GE-167) of 9% (w/w) and the weight ratios of PNVA GE-167 to aluminium chloride and of propylene glycol to N-methyl-2-pyrrolidone were kept at 1.5:1 and 2:1, respectively.

### Examples 2-5

The compositions of the matrix consisted of PNVA GE-167 with different amount of 5% (Example 2), 7% (Example 3), 11% (Example 4) and 13% (Example 5), while the weight ratios of PNVA GE-167 to aluminium chloride at 1.5:1 and of propylene glycol to N-methyl-2-pyrrolidone at 2:1 were maintained constant.

### Examples 6-9

The compositions of the matrix consisted of 9% (w/w) of PNVA GE-167 and the weight ratio of propylene glycol to N-methyl-2-pyrrolidone was maintained at 2:1 but the weight ratios of PNVA GE-167 to aluminium chloride varied from 2.5:1 (Example 6), 2:1. (Example 7), 1:1 (Example 8) to 1:1.5 (Example 9).

### Examples 10-13

The compositions of the matrix consisted of 9% (w/w) of PNVA GE-167 and the weight ratio of PNVA GE-167 to aluminium chloride was maintained at 1.5:1 but the weight ratios of propylene glycol to N-methyl-2-pyrrolidone varied from 1:2 (Example 10), 1:1 (Example 11), 3:1 (Example 12) to 4:1 (Example 13).

### Examples 14-16

The compositions of the matrix consisted of PNVA GE- 167 of 9% (w/w) and the weight ratios of PNVA GE- 167 to aluminium chloride and of propylene glycol to N-methyl-2-pyrrolidone were kept at 1.5:1 and 2:1, respectively. The amounts of aspirin per square centimeters of plasters varied from 3.4 mg (Example 14), 5.1 mg (Example 15) to 6.8 mg (Example 16).

### Experiment 1

Drug Release and Permeation Studies

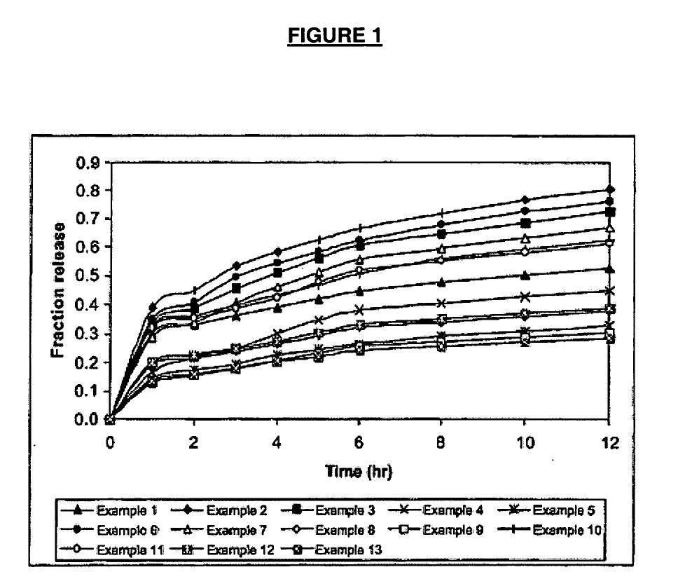

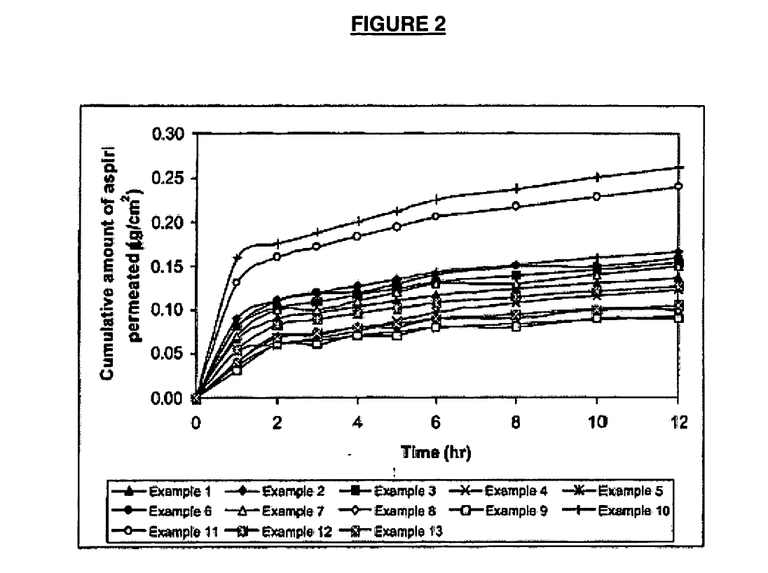

Franz vertical diffusion cells were used in the drug release and permeation studies. Aspirin plasters were mounted between the donor compartment and the receptor compartment. Cellulose filtration membranes with pore size of 0.45 μm were used in the release studies while the epidermis of porcine skins were used in the permeation studies as permeation barriers. The receptor compartment was filled with 7 ml of pH 7.4 phosphate buffer solution at 32° C. which was kept stirring at 100 rpm over the course of the experiment. The area of the plaster available for permeation was 1.766 cm². At pre-determined time intervals over a period of 12 h, 1.5 ml of samples were withdrawn from the receptor compartment with replacement of an equal volume of fresh pH 7.4 phosphate buffer solution added to maintain a constant volume. The samples obtained were analyzed by high performance liquid chromatography and cumulative amounts of drug-release or permeated were calculated. All the experiments were repeated at least six times and the results were averaged. The results are shown in FIGS. 1 and 2.

### Experiment 2

Primary Skin Irritation Studies

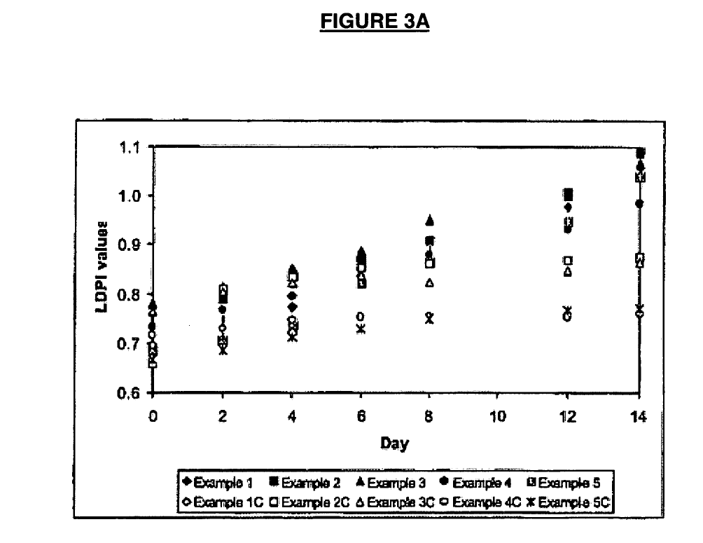

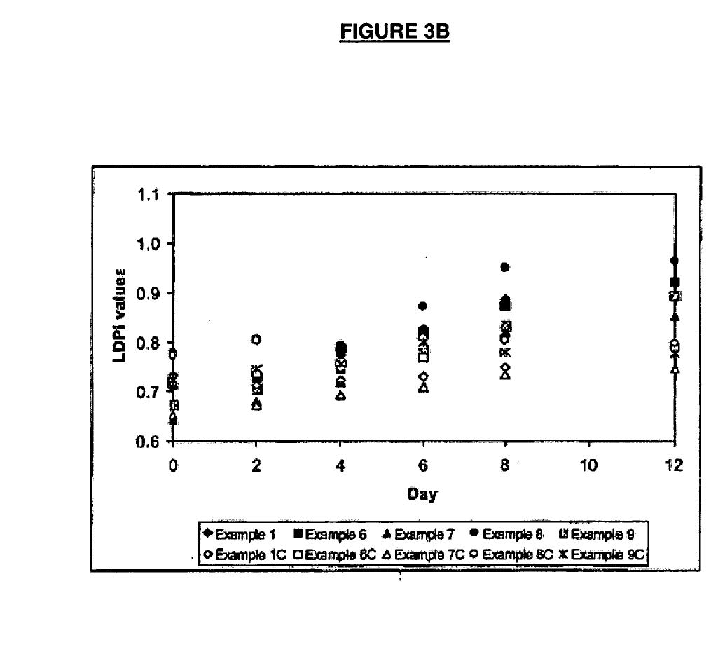

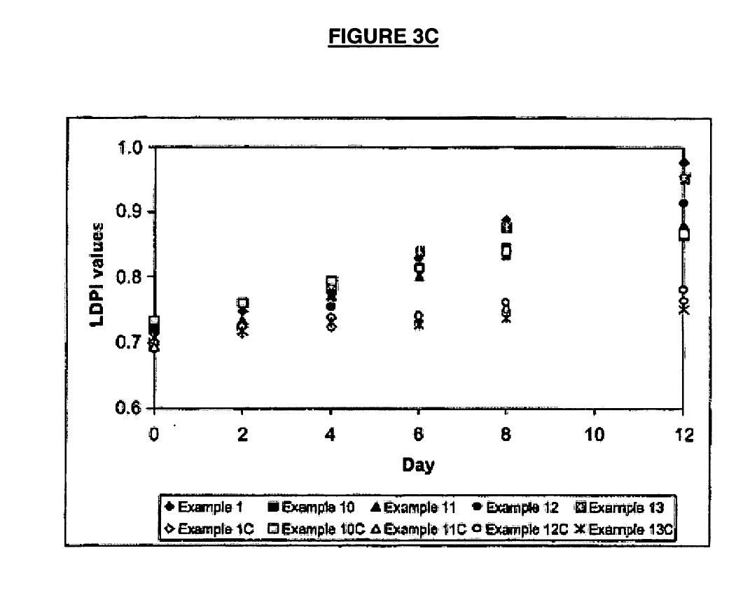

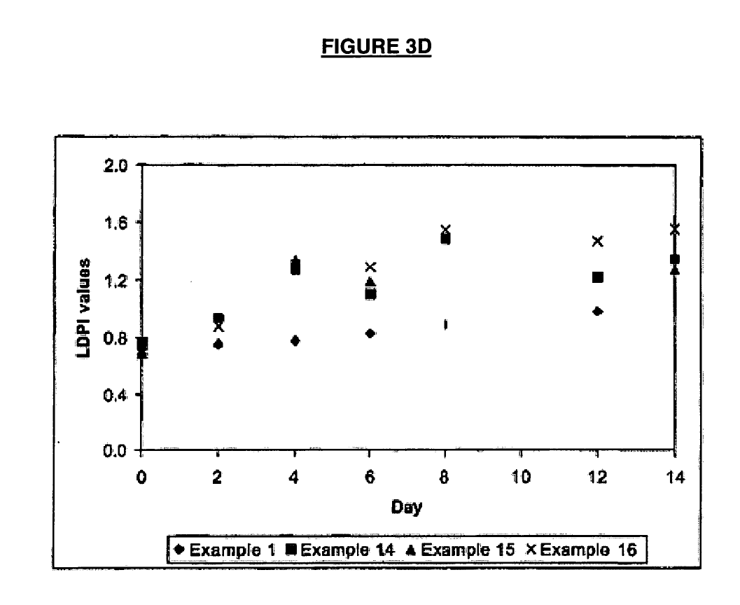

The dorsal of healthy Yorkshire pigs were clipped free of hair at least one day prior to the commencement of the experiment. Care was taken to avoid abrading the skin. For each pig, placebo plasters were applied on one side and aspirin plasters were applied on the other side of the back. All test sites were covered with surgical gauze. The pigs were housed at the standard environment with room temperature of 25° C. and relative humidity of 60%. The condition of the skin was evaluated by visual analogue score method and the cutaneous blood flow of the test sites were measured by Laser Doppler Perfusion Imager (LDPI) every two days over 14 days after removal of the plasters. After each measurement, new plasters for each composition were applied on the same application sites of the porcine skins over 2 days for further measurements. The results are shown in FIGS. 3A to 3D.

### Experiment 3

Bioadhesion Studies

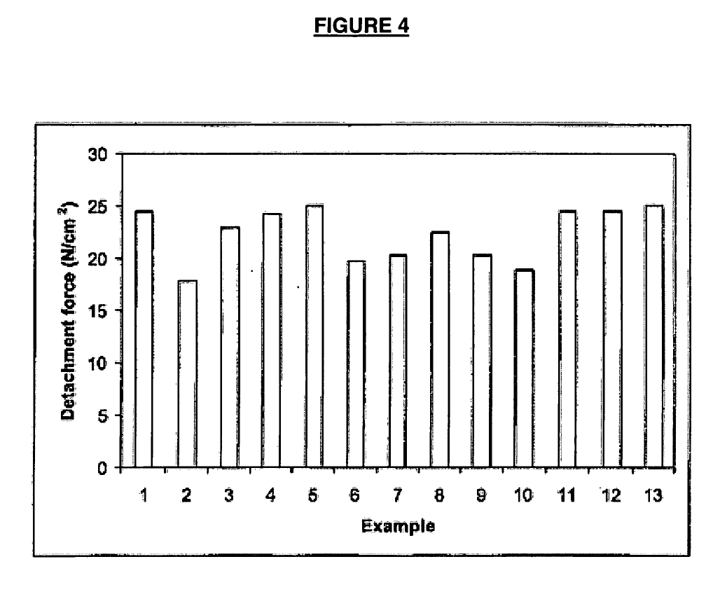

The bioadhesive properties of films were investigated by using a tensile tester (Shimadzu, EZ tester, Japan) in the tensile mode. The plaster was cut into strips of 2.0×2.0 cm² size. The strips were adhered to the lower stationary compression plate of the tensile tester with double-sided adhesive tapes. The model substrate (silicone elastomer, ISP, US), which was used to simulate the skin in terms of bioadhesion, was mounted onto the upper plate. The upper plate with the substrate attached was then lowered to the surface of the plaster and allowed to keep in contact with the plaster for 120 s under load of 10 N. It was then raised at a speed of 100 mm/min to detach the substrate from the plaster. The detachment forces, which represented the bioadhesive properties of the plasters in this study, were recorded. Six strips were evaluated for each plaster and the results averaged. The adhesive properties of plasters on porcine skins were also evaluated in vivo by scoring method (Hill Top Research, Inc., East Brunswick) and the adhesion scores were recorded. The results are shown in FIG. 4.

### Experiment 4

Rheological Measurements

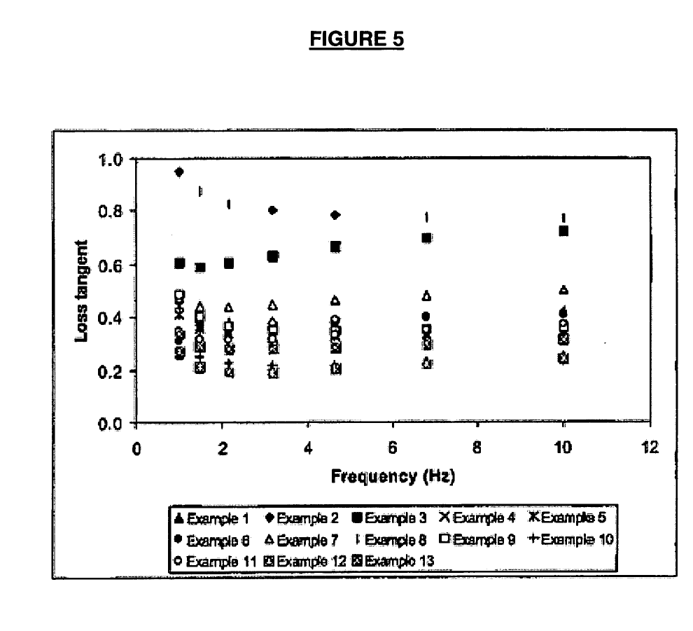

Oscillatory rheological measurements were carried out by using the rheometer (RheoStress® 1 Haake, Germany) in a cone-plate sensor system with a diameter of 35 mm and cone angle of 1′. Samples were applied to the lower plate and allowed to equilibrate to the measurement temperature at 25° C. The linear viscoelastic regions for the gels were determined by torque sweep studies from 0.5 to 500 Pa at frequencies of 1 Hz. A torque value representative of the linear viscoelastic region was chosen for the frequency sweep analysis. The oscillatory measurements were performed over a frequency range of 1 to 10 Hz at a stress of 10 Pa at 25° C. Rheological parameters including the elastic modulus G′, viscous modulus G″ and loss tangent tan 6 were obtained, respectively. Each datum point of FIG. 5 was the mean of at least three measurements.

### Experiment 5

Stability Studies

The stability studies were carried out in accordance with the ICH guidelines. The results show the stability of aspirin in the formulations.

FIG. 1 and FIG. 2 show the drug release profiles from the plasters through filtration membranes and porcine skin epidermis in pH 7.4 phosphate buffer solution at 32° C., respectively.

FIG. 3 shows the results obtained from LDPI measurement after application of the compositions of the matrix described in Examples 1-16 on porcine skin for 14 days. Essentially, the placebo did not significantly increase the cutaneous blood flow of pigs but aspirin-loaded test plasters slightly increased LDPI values, particularly for plasters with high doses of aspirin (Example 16). These implied that the increase in blood flow at the application sites was caused by aspirin itself instead of the plaster base. The primary irritation scores assessed by VAS method were zeros for Example 1-13, indicating that there are no irritation responses. The examples tested are neither corrosive nor primary irritant.

FIG. 4 shows detachment forces of the plasters comprising the composition of the matrix described in Examples 1-13 to a model skin-mimic substrate, silicone elastomer. Table 1 shows a summary of the adhesion scores after application of the composition of the matrix described in Examples 1-13 on porcine skin for 14 days. The plasters show good bioadhesive properties and can be adhered onto the skin for a long time.

TABLE 1

| | Day | | | | | | |
|---|---|---|---|---|---|---|---|
| Example | 0 | 2 | 4 | 6 | 8 | 12 | 14 |
| Example 1 | 0 | 0 | 0 | 0 | 0 | 0 | 0 |
| Example 2 | 2 | 2 | 2 | 2 | 2 | 2 | 2 |
| Example 3 | 1 | 1 | 1 | 1 | 1 | 1 | |
| Example 4 | 0 | 0 | 0 | 0 | 0 | 0 | 0 |
| Example 5 | 0 | 0 | 0 | 0 | 0 | 0 | 0 |
| Example 6 | 2 | 2 | 2 | 2 | 2 | 2 | 2 |
| Example 7 | 1 | 1 | 1 | 1 | 1 | 1 | 1 |
| Example 8 | 0 | 0 | 0 | 0 | 0 | 0 | 0 |
| Example 9 | 0 | 0 | 0 | 0 | 0 | 0 | 0 |
| Example 10 | 1 | 1 | 1 | 1 | 1 | 1 | 1 |
| Example 11 | 2 | 2 | 2 | 2 | 2 | 2 | 2 |
| Example 12 | 0 | 0 | 0 | 0 | 0 | 0 | 0 |
| Example 13 | 0 | 0 | 0 | 0 | 0 | 0 | 0 |

Adhesion scores:
0 = 90% adhered (essentially no lift off the skin);
1 = 75% to <90% adhered (some edges only lifting off the skin);
2 = 50% to <75% adhered (less than half of the system lifting off the skin);
3 = <50% adhered but not detached (more than half the system lifting off the skin without falling off);
4 = patch detached (patch completely off the skin)

FIG. 5 indicates that the compositions are cross-linked networks in nature. These results show that the delivery system of the present invention are bioadhesive hydrogel-based plasters and are promising as drug delivery carriers for water-sensitive drugs at high doses.

1. A topical and/or transdermal bioactive compound delivery system comprising a non-aqueous matrix, the matrix comprising a water-sensitive bioactive compound.

2. The delivery system according to claim 1, wherein the matrix is prepared by mixing: at least one polymer, at least one cross-linking agent, and at least one non-aqueous and/or non-volatile solvent, and wherein the non-aqueous and/or non-volatile solvent comprises the bioactive compound.

3. The delivery system according to claim 2, wherein the matrix comprises a three-dimensional polymeric network comprising at least a co-polymer, at least a cross-linking agent, and at least a non-aqueous and/or non-volatile solvent, and wherein the water-sensitive bioactive compound is housed by the three-dimensional network.

4. The delivery system according to claim 3, wherein the three-dimensional network is hydrogel.

5. The delivery system according to claim 2, wherein the at least one polymer is a polymer or co-polymer selected from: polymer of N-vinylacetamide, polymer of acrylic acid, polymer of acrylate, co-polymer of acrylic acid and sodium acrylate.

6. The delivery system according to claim 2, wherein the cross-linking agent is selected from an aluminium salt and trivalent salt.

7. The delivery system according to claim 2, wherein the cross-linking agent is aluminium chloride.

8. The delivery system according to claim 2, wherein the solvent is N-methyl-2-pyrrolidone.

9. The delivery system according to claim 2, wherein the matrix is prepared by mixing: (a) a co-polymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

10. The delivery system according to claim 1, wherein the bioactive compound is a drug.

11. The delivery system according to claim 10, wherein the drug is aspirin.

12. The delivery system according to claim 1, wherein the delivery system is a bioadhesive topical and/or transdermal delivery system.

13. The delivery system according to claim 1, wherein the delivery system is in the form of a plaster.

14. The delivery system according to claim 13, wherein the plaster comprises a backing material.

15. The delivery system according to claim 14, wherein the backing material comprises at least one material selected from polyester, polypropylene, and polyethylene.

16. A bioadhesive topical and/or transdermal bioactive compound delivery system comprising a non-aqueous matrix, wherein the matrix is prepared by mixing: (a) a copolymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

17. The delivery system according to claim 16, wherein the delivery system is in the form of a plaster.

18. A method of preparing a topical and/or transdermal bioactive compound delivery system comprising preparing a polymeric non-aqueous matrix, the matrix comprising a water-sensitive bioactive compound.

19. The method according to claim 18, wherein the matrix is prepared by mixing: at least one polymer, at least one cross-linking agent, and at least one non-aqueous and/or non-volatile solvent, and wherein the non-aqueous and/or non-volatile solvent comprises the bioactive compound.

20. The method according to claim 18, wherein the matrix is prepared by mixing (a) a copolymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

21. A method of topical and/or transdermal administration of a water-sensitive bioactive material, comprising preparing a polymeric non-aqueous matrix, the matrix comprising a water-sensitive bioactive compound, and applying the matrix on the skin of a mammal in need of treatment.

22. The method according to claim 21, wherein the matrix is prepared by mixing: at least one polymer, at least one cross-linking agent, and at least one non-aqueous and/or non-volatile solvent, and wherein the non-aqueous and/or non-volatile solvent comprises the bioactive compound.

23. The method according to claim 21, wherein the matrix is prepared by mixing (a) a copolymer of N-vinylacetamide and sodium acrylate; (b) aluminium chloride; (c) N-methyl-2-pyrrolidone; (d) propylene glycol; (e) glycerine; and (f) at least a water-sensitive bioactive compound.

24. The method according to claim 21, wherein the topical and/or transdermal administration is in the form of a plaster or patch.

25. The method according to claim 21, wherein the bioactive compound is a drug.
